(19) United States
(12) Patent Application Publication
(10) Pub. No.: US 2026/0198907 A1
(43) Pub. Date: Jul. 16, 2026

(54) Nasal swab sampling robot with force feedback member and sampling method

(71) Applicant: THE FIRST AFFILIATED HOSPITAL OF GUANGZHOU MEDICAL UNIVERSITY (GUANGZHOU RESPIRATORY CENTER), Guangzhou (CN)

(72) Inventors: Shaoqiang LI, Guangzhou (CN); Jianqing HAO, Guangzhou (CN); Weilong LI, Guangzhou (CN); Chengzhi ZHOU, Guangzhou (CN)

(21) Appl. No.: 19/361,657

(22) Filed: Oct. 17, 2025

Related U.S. Application Data
(63) Continuation of application No. PCT/CN2025/077624, filed on Feb. 17, 2025.

(30) Foreign Application Priority Data
Jan. 16, 2025 (CN) 202510069174.1

(51) Int. Cl. A61B 10/02; A61B 90/00

(52) U.S. Cl. CPC A61B 10/02; A61B 90/06; A61B 2090/064

(57) ABSTRACT

A nasal swab sampling robot includes a nasal swab sampling head; a force feedback member connected to the nasal swab sampling head, which is configured to automatically telescopically adjust the nasal swab sampling head for sampling; and a multi-directional actuating member connected to the nasal swab sampling head, which is configured for multi-directional displacement adjustment of the nasal swab sampling head. The multi-directional actuating member includes a forward and backward moving mechanism connected to the nasal swab sampling head via the force feedback member, a vertical moving mechanism, a pitch angle swinging mechanism, and a circumferential angle swinging mechanism. The forward and backward moving mechanism, the pitch angle swinging mechanism, and the circumferential angle swinging mechanism are all connected to the vertical moving mechanism for adjusting a combined trajectory of a vertical displacement, a circumferential swinging displacement, and a pitch angle swinging displacement of the nasal swab sampling head.

### CROSS REFERENCE TO RELATED APPLICATIONS

The present application is a continuation of International Application No. PCT/CN2025/077624, filed on Feb. 17, 2025, which claims priority from Chinese Application No. 202510069174.1 filed on Jan. 16, 2025, all of which are hereby incorporated herein by reference.

### TECHNICAL FIELD

The present disclosure relates to the field of medical instruments, and in particular, to a nasal swab sampling robot with a force feedback member, and a sampling method.

### BACKGROUND

During operation of a nasal swab, a medical person is required to be in close contact with a patient, and during sampling a large number of droplets or aerosols may be produced when the patient coughs and breathes hard. A sampling worker (medical person) is also in close contact with a nasal swab sampling object (patient), especially when a nasal swab is inserted into a nostril, the patient, stimulated by the swab, is prone to sneezing, increasing the risk of breakage of the nasal swab in the nasopharynx and posing significant risks to the sampling worker. Furthermore, differences in the skill levels of medical persons and non-standardized nasal swab sampling operations would lead to discrepancies in swab quality, which further affects the diagnosis on patient's conditions.

The current sampling device generally relies on manual operation. During manual sampling, it is difficult to control the force applied, resulting in obvious discomfort when the patient is sampled. Moreover, non-standardized sampling operations may lead to noticeable differences in swab quality, which further affects the accuracy of sample collection.

Therefore, it is desirable to standardize sampling operation for different patients and accurately control the sampling force of each sampling to avoid unstable swab quality caused by manual operation, which could otherwise affect sampling efficiency.

### SUMMARY

The present disclosure provides a nasal swab sampling robot with a force feedback member and a sampling method, which can standardize sampling operations for different patients, and accurately controlling the sampling force of each sampling to avoid unstable swab quality caused by manual operation, which could otherwise affect sampling efficiency.

In a first aspect, the present disclosure provides a nasal swab sampling robot, including:
a nasal swab sampling head;
a force feedback member connected to the nasal swab sampling head, which is configured to automatically telescopically adjust the nasal swab sampling head for sampling; and
a multi-directional actuating member connected to the nasal swab sampling head, which is configured for multi-directional displacement adjustment of the nasal swab sampling head,
where the multi-directional actuating member includes a forward and backward moving mechanism, a vertical moving mechanism, a pitch angle swinging mechanism, and a circumferential angle swinging mechanism, where the forward and backward moving mechanism is connected to the nasal swab sampling head via the force feedback member, and the forward and backward moving mechanism, the pitch angle swinging mechanism and the circumferential angle swinging mechanism are all connected to the vertical moving mechanism for adjusting a combined trajectory of a vertical displacement, a circumferential swinging displacement, and a pitch angle swinging displacement of the nasal swab sampling head.

In a second aspect, the present disclosure provides a sampling method, including steps of:
S1) determining, by a vision device, whether a patient is present in front of the sampling robot, if not, maintaining the sampling robot in a powered-off state, and if yes, switching the sampling robot to a standby state;
S2) determining, by the vision device, a nostril position to be sampled according to images of patients of different heights and ages in front of the robot, and sending an instruction to control the forward and backward moving mechanism, the vertical moving mechanism, and the circumferential angle swinging mechanism to perform a rough adjustment at entrance of the nostril position;
S3) determining, by the vision device, a nasal bridge position to be sampled according to the images, calculating an inclination angle of the nostril close to the nasal bridge position, and automatically sending an instruction to control the pitch angle swinging mechanism to perform a rough adjustment in a direction parallel to the inclination angle;
S4) inserting the nasal swab sampling head into the nostril after the rough adjustment, and sending a feedback instruction to the pitch angle swing mechanism, the forward and backward moving mechanism, and the circumferential angle swing mechanism to perform a fine adjustment according to resistance experienced by the force feedback member in a nasal cavity; and
S5) performing sampling upon reaching a sampling position in the nasal cavity, and withdrawing from the nasal cavity along the direction parallel to the inclination angle in S3 after sampling is completed.

### BRIEF DESCRIPTION OF THE DRAWINGS

FIG. 1a shows a front view of a nasal swab sampling robot according to the present disclosure;

FIG. 1b shows a side view of the nasal swab sampling robot;

FIG. 1c shows a top view of the nasal swab sampling robot;

FIG. 2 shows a circumferential angle swinging mechanism of the nasal swab sampling robot according to the present disclosure;

FIG. 3 shows an assembly of a circumferential angle swing mechanism, a pitch angle swinging mechanism, and a vertical moving mechanism of the nasal swab sampling robot according to the present disclosure;

FIG. 4 is a view showing a forward and backward moving mechanism of the nasal swab sampling robot according to the present disclosure;

FIG. 5 is another view of the forward and backward moving mechanism;

FIG. 6 is a sectional view of the forward and backward moving shown in FIG. 4;

FIG. 7a is a perspective view of the nasal swab sampling robot including a positioning mechanism according to the present disclosure, with a limiting frame removed; and

FIG. 7b is another perspective view of the nasal swab sampling robot including a positioning mechanism according to the present disclosure.

### DETAILED DESCRIPTION OF THE EMBODIMENTS

The accompanying drawings of the present disclosure are only intended for illustrative purposes and should not be construed as limiting the present disclosure. In order to better illustrate the following embodiments, some components in the accompanying drawings may be omitted, enlarged or reduced, and do not represent actual product dimensions. It will be understood by those skilled in the art that certain well-known structures and their descriptions in the accompanying drawings may be omitted.

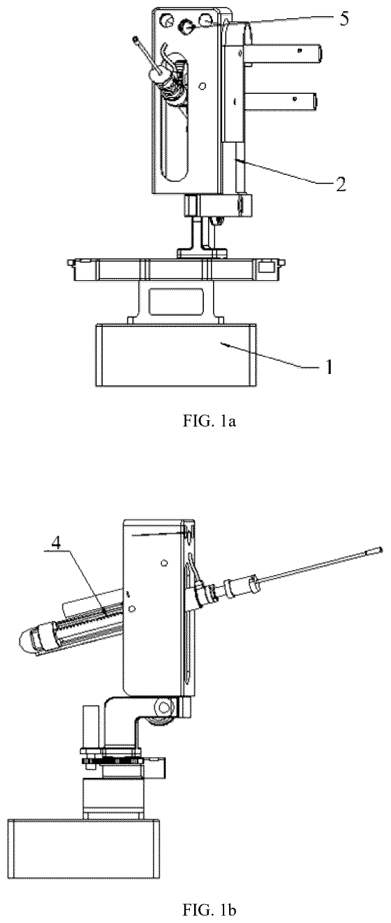

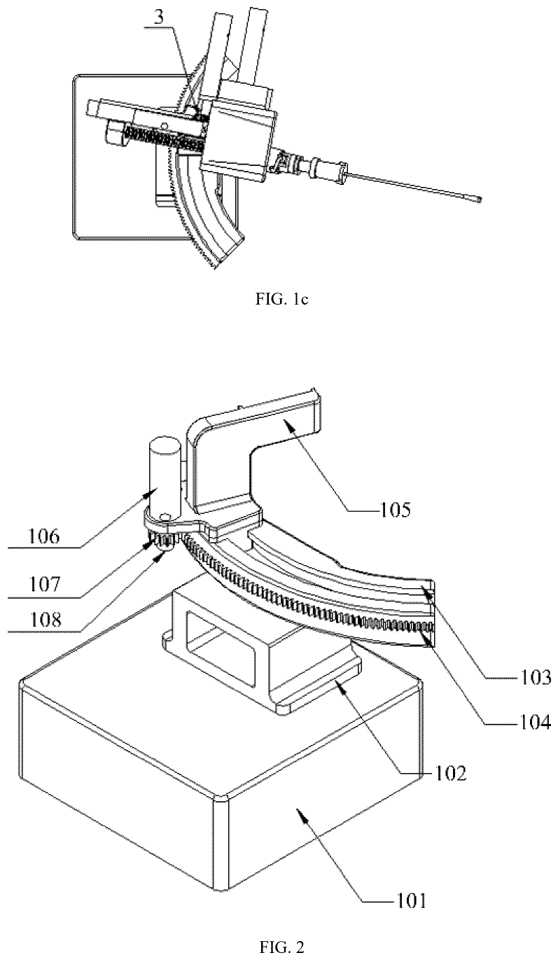

FIGS. 1a to 1c provide a nasal swab sampling robot, including a multi-directional actuating member, a nasal swab sampling head 315, and a force feedback member. The nasal swab sampling head 315 is connected with the force feedback member to achieve automatic telescopic adjustment for sampling, and connected with the multi-directional actuating member to achieve multi-directional displacement adjustment. Particularly, the multi-directional actuating member includes a circumferential angle swinging mechanism 1, a vertical moving mechanism 2, a pitch angle swinging mechanism 3, and a forward and backward moving mechanism 4. The nasal swab sampling head 315 is connected to the forward and backward moving mechanism 4 via the force feedback member. The circumferential angle swinging mechanism 1, the pitch angle swinging mechanism 3, and the forward and backward moving mechanism 4are all connected to the vertical moving mechanism 2 to adjust a combined trajectory of the pitch angle swinging displacement, circumferential swinging displacement, and vertical displacement, of the nasal swab sampling head 315.

In this embodiment, the multi-directional actuating member of the sampling robot is configured to control the multi-directional movement of the nasal swab sampling head 315, so as to achieve precise sampling positions of different patients, and the force feedback member is configured to detect whether the sampling force exceeds a threshold value, if the sampling force exceeds the threshold value, automatic rebound of the nasal swab sampling head 315 is conducted, which thus can control sampling force to reduce discomfort experienced by the patients in the sampling process.

In the present embodiment, the mounting direction of the nasal swab sampling head 315 is denoted as a front-back direction, a direction perpendicular to the mounting direction of the nasal swab sampling head 315 is denoted as a vertical direction, and the swinging direction along the vertical plane and the swinging direction the horizontal plane perpendicular to the vertical plane, when the nasal swab sampling head 315 is fixed in one vertical position, are respectively denoted as a pitch direction and an annular swinging direction. Accordingly, the forward and backward moving mechanism 4 controls the movement of the nasal swab sampling head 315 in the front-back direction, the vertical moving mechanism 2 controls the movement of the nasal swab sampling head 315 in the vertical direction, the pitch angle swinging mechanism 3 controls the movement of the nasal swab sampling head 315 in the pitch direction, and the circumferential angle swinging mechanism 1 controls the movement of the nasal swab sampling head 315 in the annular swinging direction.

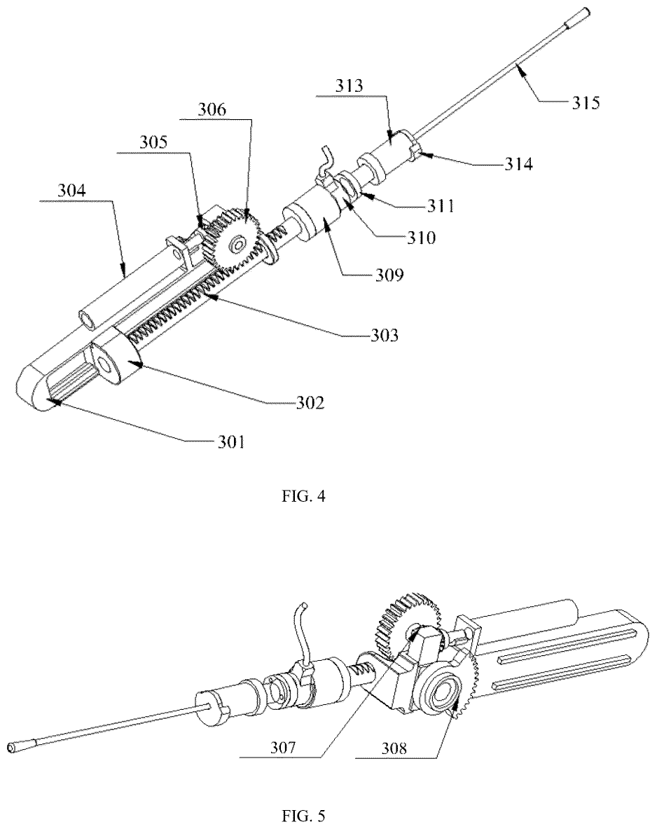

Now referring to FIG. 4, the forward and backward moving mechanism 4 particularly includes a telescopic frame 301, a telescopic block 302, a rack shaft 303, and a gear 306. The gear 306 is fixedly connected to the telescopic frame 301 and meshed with the rack shaft 303. One end of the rack shaft 303 is connected with the telescopic block 302, and the other end of the rack shaft 303 is connected with the nasal swab sampling head 315. The gear 306 is rotated by a first motor 304 to drive the rack shaft 303 to move, which in turn drives the telescopic block 302 to move in the telescopic frame 301 and drives the nasal swab sampling head 315 to move forward and backward.

The telescopic frame 301 in the present embodiment is used to limit the direction and trajectory of the telescopic displacement of the telescopic block 302 which allows the nasal swab sampling head 315 to move forward and backward. The cooperation of the rack shaft 303 and the gear 306 can achieve accurate control of forward and backward displacement of the nasal swab sampling head 315.

In this embodiment, the telescopic frame 301 of the forward and backward moving mechanism 4 is internally provided with a groove, especially a dovetail groove, and the telescopic block 302 is mounted in the dovetail groove and moves forward and backward along the groove. The first motor 304 and the forward and backward rear rotating gear 306 are fixed outside the telescopic frame 301. One end of the rack shaft 303 is fixedly connected with the nasal swab sampling head 315, and the other end is fixedly connected with the telescopic block 302. The teeth of the rack shaft 303 are meshed with the gear 306 at the upper end. Therefore, when the first motor 304 drives the gear 306 to rotate, the rack shaft 303 drives the telescopic block 302 and the nasal swab sampling head 315 to move in the front-back direction respectively under the meshing action.

In combination with FIG. 5, an output shaft of the first motor 304 is provided with a driving bevel gear 305, an end of the telescopic frame 301 is provided with a driven bevel gear 307, the driving bevel gear 305 is connected with the driven bevel gear 307 via a bearing, and the driven bevel gear 307 is coaxially connected with the gear 306. As a result, the driving bevel gear 305 driven by the first motor 304 drives the driven bevel gear 307 to rotate, which in turn drives the gear 306 to rotate.

With configuration of the driving bevel gear 305 and the driven bevel gear 307, the output shaft of the first motor 304 and the gear 306 keeps parallel to the rack shaft 303, which avoids collision between the first motor 304 and other mechanisms when the nasal swab sampling head 315 moves forward and backward, as well as reducing the volume of the sampling robot. In addition, bearing connection between the driving bevel gear 305 and the driven bevel gear 307 can achieve high-precision rotation transmission between the output shaft of the first motor 304 and the gear 306.

In this embodiment, the upper end of the telescopic frame 301 is fixedly connected with the first motor 304 through a flange. The driving bevel gear 305 is mounted on the output shaft of the first motor 304 by a jacking screw. A bearing hole is formed at a position 90 degrees relative to the position where the bevel gear 305 is mounted on the telescopic frame 301, the bearing is mounted in the bearing hole, and a driven bevel gear 307 shaft is mounted in the bearing. The two bevel gears are meshed each other to achieve relative rotation. The gear 306 is fixedly mounted at the other end of the driven bevel gear 307 so as to rotate coaxially with the gear shaft of the bevel gear 307.

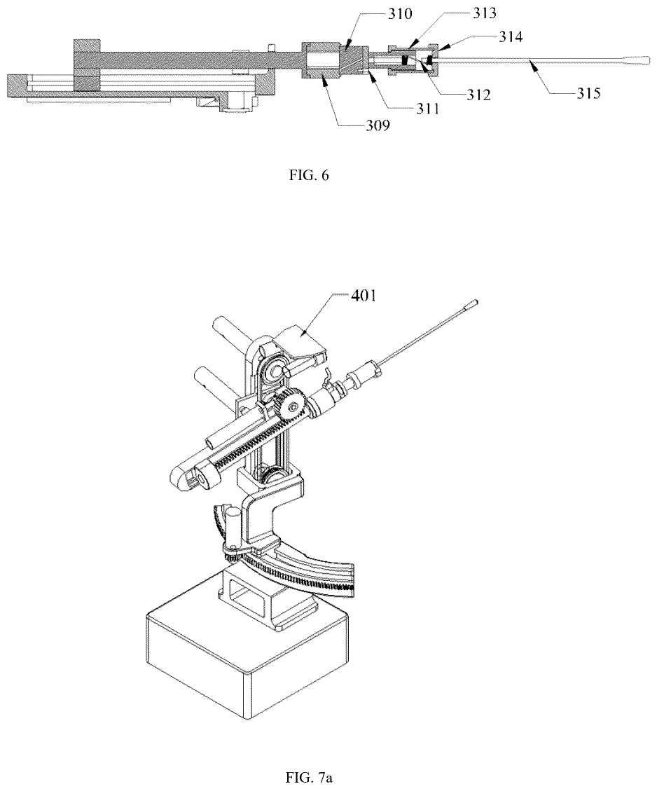

With reference to FIG. 4 and FIG. 6, the force feedback member in the present embodiment particularly includes a fixed end support 309, a movable end support 311, and a pressure sensor 310. An end of the rack shaft 303 is fixedly connected with the fixed end support 309, the nasal swab sampling head 315 is movably connected with the movable end support 311 via a telescopic sleeve 313, and the pressure sensor 310 is arranged between the fixed end support 309 and the movable end support 311.

In such configuration, the acting force for the forward and backward movement of the nasal swab sampling head 315 is transmitted to the movable end support 311 through the telescopic sleeve 313, so that corresponding force in the movable end support 311 can be detected by the pressure sensor 310. The fixed end support 309 provides a support for the pressure sensor 310. In this way, the nasal swab sampling head 315 is retracted to avoid harm to the patient when excessive extrusion force is applied.

In this embodiment, the fixed end support 309, the pressure sensor 310 and the movable end support 311 are successively fixed in the axial direction of the rack shaft 303. To ensure coaxial connection thereof, the telescopic sleeve 313 is mounted outside the movable end support 311, so that the telescopic sleeve 313 and the movable end support 311 are allowed to slide relative to each other. A compression spring 312 is mounted between the inner end face of the movable end support 311 and the inner end face of the telescopic sleeve 313, as shown in FIG. 6. A sampling head coupling cover 314 is mounted at the front end of the telescopic sleeve 313, and the two may be quickly snap-fitted. The nasal swab sampling head 315 is fixedly mounted at the foremost end of the sampling head coupling cover 314. When the first motor 304 is activated, a linear movement function is completed through the bevel gear transition transmission to a gear-rack structure group. This enables the linear telescopic function of the nasal swab sampling head 315. In addition, under the action of the pressure sensor 310 and the compression spring 312, the nasal swab sampling head 315 can be automatically retracted if extending too far when inserted into the nostril and undergoing too large resistance, which prevents harm to a sampled person due to excessive sampling force.

In this embodiment, the pressure sensor 310 converts a pressure difference between the fixed end support 309 and the movable end support 311 into an electrical signal, and sends the electrical signal to the first motor 304 through a wireless transmitter. When the pressure difference increases, the electrical signal increases, and the first motor 304 drives the driving bevel gear 305 to rotate, thereby driving the gear 306 to move in a direction close to the fixed end support 309, causing the nose swab sampling head 315 to retract.

Referring back to FIG. 3, the vertical moving mechanism 2 in the present embodiment particularly includes a sliding support 207 and a movable seat 210. The movable seat 210 is vertically slidable in the sliding support 207, and the pitch angle swinging mechanism 3 is connected to the sliding support 207 via the movable seat 210.

Additionally, the sliding support 207 includes a vertical groove, a driving rotating wheel 208, a synchronous belt 203, a driven rotating wheel 201, and a positioning rotating gear 205. The movable seat 210 includes a clamping portion, a transition portion, and a gear portion 308. A side surface of the movable seat 210 is fixedly attached to the synchronous belt 203, the clamping portion is slidably clamped in the vertical groove, the positioning rotating gear 205 is meshed with the gear portion 308, and the transition portion is rotatably connected to the forward and backward moving mechanism 4. The movable seat 210 is driven to rotate in the pitch direction by the positioning rotating gear 205 through a second motor 204. The driving rotating wheel 208 is driven by a third motor 206 so as to drive the driven rotating wheel 201 to rotate under the driving of the the synchronous belt 203, so that the movable seat 210 is driven to move in the vertical groove by the synchronous belt 203.

In such configuration, the sliding support 207 provides fixation of the robot in the vertical direction, and the displacement of the nasal swab sampling head 315 in the vertical direction is achieved by the movable seat 210. With connection of the pitch angle swinging mechanism 3 and the sliding support 207, a combined trajectory of the pitch angle swinging and vertical movement of the nasal swab sampling head 315 is allowed, which can standardize each sampling step, thereby achieving accurate sampling.

In this embodiment, the positioning rotating gear 205 and the gear portion 308 form at least portion of the pitch angle swinging mechanism 3. With the connection between the pitch angle swinging mechanism 3 and the movable seat 210, a movement trajectory of the movable seat 210 in the vertical direction is combined with a movement trajectory of the pitch angle swinging mechanism 3 in the pitch direction, making the sampling movement trajectory more flexible to accommodate different sampling positions.

In this embodiment, the third motor 206 is mounted at an upper end flange of the sliding support 207 by screws. A driving end coupling shaft 209 is mounted at the output end of the third motor 206 by screws, especially set screws. The driving rotating wheel 208 is mounted on the driving end coupling shaft 209 and located at a shaft shoulder of the driving end coupling shaft 209 in the axial direction, especially through screws, particularly set screws, so that the driving rotating wheel 208 is driven to rotate by the third motor 206. The lower end of the sliding support 207 is provided with a driven end coupling shaft 211 and the driven rotating wheel 201, enabling rotation of the driven rotating wheel 201 relative to the sliding support 207. During mounting, it is required to ensure that the driven rotating wheel 201 and the driving rotating wheel 208 are coplanar at the axial end face, and ensure that the transmission of the synchronous belt 203 between the driving rotating wheel 208 and the driven rotating wheel 201 maintains stable when the third motor 206 drives the synchronous belt 203 to rotate.

In this embodiment, the sliding support 207 is internally provided with the vertical groove which is preferably dovetail-shaped, and the movable seat 210 is slidably mounted in the vertical groove. The side surface of the movable seat 210 and the side surface of the synchronous belt 203 are fixed into a whole by clamping screws. In such configuration, when the third motor 206 drives the driving rotating wheel 208 to rotate, the movable seat 210 is driven to move upward and downward between the two rotating wheels along the vertical groove under driving of the synchronous belt 203, thereby achieving precise control of the vertical movement of the nasal swab sampling head 315. The second motor 204 is mounted at a flange of the movable seat 210. The positioning rotating gear 205 is mounted on an output shaft of the second motor 204 and connected with the second motor 204 by screws. An and of the output shaft of the second motor 204 is provided with a bearing, the gear portion 308 is mounted at an outer ring of the bearing and connected with the bearing via a snap ring in the axial direction, allowing the gear portion 308 to rotate relative to the movable seat 210 in the radial direction. In this way, the end face of the positioning rotating gear 205 is integrally linked with the forward and backward moving mechanism 4 including the nasal swab sampling head 315, and the positioning rotating gear 205 is meshed with the gear portion 308, so that the gear portion 308 is driven to rotate through the meshing action during rotation of the positioning rotating gear 205 driven by the second motor 204, and the gear portion 308 in turn drives the movable seat 210 connected to the forward and backward moving mechanism 4 including the nasal swab sampling head 315 to swing in the pitch direction, thus achieving a pitch swinging movement function of the nasal swab sampling head 315.

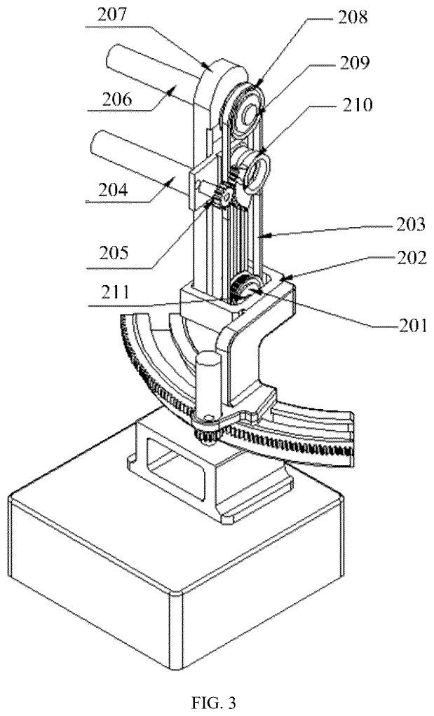

With reference to FIG. 2 and FIG. 3, the circumferential angle swinging mechanism 1 includes a circumferential guide rail, a circumferential movable block 105, and a rotating gear 107, and the vertical moving mechanism 2 includes a fixed seat 202. The fixed seat 202 is usually fixed at the bottom of the sliding support 207, especially the top of the circumferential movable block 105 is connected to the sliding support 207 via the fixed seat 202, and the bottom of the circumferential movable block 105 slides along the circumferential guide rail through the rotating gear 107.

In this embodiment, the fixed seat 202 is particularly frame-shaped, and the sliding support 207 extends through the frame-shaped fixed seat 202 and fixedly connected with the fixed seat 202. The upper portion of the circumferential movable block 105 is preferably L-shaped, the top end of the L-shaped circumferential movable block 105 is fixed to the fixed seat 202, and the bottom end moves along the circumferential guide rail. The rotating gear 107 is fixedly connected with the circumferential movable block 105 via a gear fixing block 108, so that when the rotating gear 107 rotates, the circumferential movable block 105 circumferentially slides along the circumferential guide rail.

As depicted in FIG. 2, the circumferential guide rail particularly includes an annular groove 103 and annular outer edge teeth 104, and the circumferential movable block 105 includes a connecting seat and a sliding block connected with each other. The rotating gear 107 is fixedly connected with the sliding block via the connecting seat, meshed with the annular outer edge teeth 104, and driven to rotate by a fourth motor 106. The rotation of the rotating gear 107 drives the connecting seat to move in a direction of the annular outer edge teeth 104, thereby driving the sliding block to slide in the annular groove 103.

In this embodiment, the annular guide rail is internally provided with the annular groove 103 which is preferably dovetail-shaped, and the circumferential movable block105 is mounted in the dovetail groove, allowing the circumferential movable block 105 to slide inside the annular groove 103. The connecting seat is formed at the upper portion of the circumferential movable block 105. The connecting seat is formed with a protrude in a lateral direction to mount the fourth motor 106. The rotating gear 107 is mounted at an output shaft of the fourth motor 106, and the annular outer edge teeth 104 are mounted at an outer edge of the annular groove 103 in a circumferential direction. The rotating gear 107 is meshed with the annular outer edge teeth 104. In such configuration, when the fourth motor 106 drives the rotating gear 107 to rotate, the rotating gear 107 drives the connecting block to move along the trajectory of the annular outer edge teeth 104 through the meshing action, which in turn drives the circumferential movable block 105 to move annularly or circumferentially in the dovetail groove, thereby achieving horizontal left-right swinging function of the nasal swab sampling head 315, namely pan movement.

As shown in FIG. 2, a guide rail supporting base 102 and a counterweight supporting base 101 are further included. The circumferential guide rail is fixed on the guide rail supporting base 102, the counterweight supporting base 101 is fixed under the guide rail supporting base 102, and a ratio of a bottom area of the counterweight supporting base 101 to a bottom area of the guide rail supporting base ranges from 1.5 to 3.

The guide rail supporting base 102 is used to raise the circumferential guide rail to a certain height, so as to prevent the sampling robot from colliding with a tabletop when pitching downward. The counterweight supporting base 101 can avoid shift caused by unstable center of gravity of the sampling robot due to movement in three directions, which could otherwise affect the sampling accuracy. In addition, the specific ratio of the bottom area of the counterweight support base 10 to the bottom area of the guide rail supporting base 102 satisfies the weight of the sampling robot through.

In this embodiment, the counterweight supporting base 101 is provided at the bottom of the circumferential angle swinging mechanism 1, with the guide rail supporting base 102 arranged at the upper end of the counterweight supporting base 101. The circumferential guide rail is fixedly mounted at a positioning portion of the guide rail supporting base 102, so that the circumferential guide rail and the guide rail supporting base 102 are kept fixed in a horizontal state. The bottom area of the counterweight supporting base 101 is preferably twice that of the guide rail supporting base.

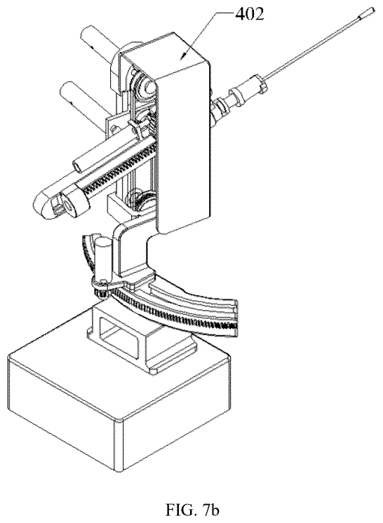

Referring to FIG. 7a and FIG. 7b, a positioning mechanism 5 is further included, which includes a limiting frame 402 and a vision device 401. The limiting frame 402 is hollowed out in a front-back direction to restrict the vertical displacement of the nasal swab sampling head 315, and the vision device 401 is arranged at the top of the limiting frame 402 to accurately position the nasal swab sampling head 315.

In this embodiment, the vision device 401 is mounted on the limiting frame 402. Both the side surface and the bottom surface of the limiting frame 402 are fixedly connected with the multi-directional actuating member. The vision device 401 includes a camera, with its focus always aligned with the end of the nasal swab sampling head 315, allowing the sampling robot to accurately position the sampling position of the patient in the sampling process and complete automatic detection of a plurality of positions of the patient. The limiting frame 402 is hollowed out in the front-back direction, also restricting the amplitude of the nasal swab sampling head 315 in the pitch direction to avoid the undesirable influence on sampling hygiene due to excessive amplitude.

A sampling method using the sampling robot mentioned above is further provided.

In step S1, whether a patient is present in front of the sampling robot is determined by the vision device 401, if not, the sampling robot is maintained in a powered-off state, and if yes, the sampling robot is switched to a standby state.

In step S2, a nostril position to be sampled is determined by the vision device 401 according to images of patients of different heights and ages in front of the sampling robot, and an instruction is sent to control the forward and backward moving mechanism 4, the vertical moving mechanism 2, and the circumferential angle swinging mechanism 1 to perform a rough adjustment at the entrance of the nostril position.

In step S3, a nasal bridge position to be sampled is determined by the vision device 401 according to the images, an inclination angle of the nostril close to the nasal bridge position is calculated, and an instruction is automatically sent to control the pitch angle swinging mechanism 3 to perform a rough adjustment in a direction parallel to the inclination angle.

In step S4, the nasal swab sampling head 315 is inserted into the nostril after the rough adjustment, and a feedback instruction is sent to the pitch angle swing mechanism 3, the forward and backward moving mechanism 4, and the circumferential angle swing mechanism 1 to perform a fine adjustment according to the resistance experienced by the force feedback member in the nasal cavity.

In step S5, rapid sampling is performed upon the nasal swab sampling head 315 reaching the sampling position in the nasal cavity, and the nasal swab sampling head 315 is withdrawn from the nasal cavity along the direction parallel to the inclination angle as mentioned in step S3 after sampling is completed.

According to the present disclosure, the multi-directional actuating member adjusts the combined trajectory of the multi-directional displacement of the forward and backward movement, vertical movement, circumferential swinging, and pitch swinging of the nasal swab sampling head 315 to accommodate the precise sampling positions of different patients. The force feedback member controls the sampling force to reduce the discomfort of patients in the sampling process. Therefore, standardized sampling is provided, with precise control of the sampling force, avoiding unstable swab quality caused by manual operation and thus improving sampling efficiency and accuracy.

Obviously, the above-mentioned embodiments of the present disclosure are only examples for clearly explaining the technical solutions of the present disclosure, and are not intended to limit the specific implementations of the present disclosure. Any modifications, equivalent substitutions or improvements made within the spirit and principle of the claims of present disclosure should be included in the protection scope of the claims of the present disclosure.

What is claimed is:

1. A nasal swab sampling robot, comprising:
a nasal swab sampling head;
a force feedback member connected to the nasal swab sampling head, which is configured to automatically telescopically adjust the nasal swab sampling head for sampling; and
a multi-directional actuating member connected to the nasal swab sampling head, which is configured for multi-directional displacement adjustment of the nasal swab sampling head,
wherein the multi-directional actuating member comprises a forward and backward moving mechanism, a vertical moving mechanism, a pitch angle swinging mechanism, and a circumferential angle swinging mechanism, wherein the forward and backward moving mechanism is connected to the nasal swab sampling head via the force feedback member, and the forward and backward moving mechanism, the pitch angle swinging mechanism, and the circumferential angle swinging mechanism are all connected to the vertical moving mechanism for adjusting a combined trajectory of a vertical displacement, a circumferential swinging displacement, and a pitch angle swinging displacement of the nasal swab sampling head.

2. The nasal swab sampling robot according to claim 1, wherein the forward and backward moving mechanism comprises:
a telescopic frame;
a telescopic block;
a rack shaft, one end of the rack shaft being connected with the telescopic block and the other end of the rack shaft being connected with the nasal swab sampling head; and
a gear fixedly connected to the telescopic frame and meshed with the rack shaft, which is rotated by a first motor to drive the rack shaft to move,
wherein the telescopic block is configured to move in the telescopic frame with movement of the rack shaft, and the nasal swab sampling head is configured to move forward and backward with movement of the rack shaft.

3. The nasal swab sampling robot according to claim 2, wherein an output shaft of the first motor is provided with a driving bevel gear, an end of the telescopic frame is provided with a driven bevel gear connected with the driving bevel gear via a bearing and coaxially connected with the gear, and the gear is configured to rotate together with the driven bevel gear which is driven by the driving bevel gear rotated by the first motor.

4. The nasal swab sampling robot according to claim 2, wherein the force feedback member comprises:
a fixed end support, fixedly connected with the rack shaft;
a movable end support, movably connected with the nasal swab sampling head via a telescopic sleeve; and
a pressure sensor, arranged between the fixed end support and the movable end support.

5. The nasal swab sampling robot according to claim 4, wherein a spring element is arranged between an inner end face of the movable end support and an inner end face of the telescopic sleeve.

6. The nasal swab sampling robot according to claim 1, wherein the vertical moving mechanism comprises:
a sliding support; and
a movable seat vertically slidable in the sliding support,
wherein the pitch angle swinging mechanism is connected to the sliding support via the movable seat.

7. The nasal swab sampling robot according to claim 6, wherein the movable seat is connected to the forward and backward moving mechanism and has a gear portion engaged with a positioning rotating gear which is driven by a second motor, the positioning rotating gear and the gear portion are formed at least portion of the pitch angle swinging mechanism, and the movable seat is configured to move in a pitch direction under cooperation of the positioning rotating gear and the gear portion.

8. The nasal swab sampling robot according to claim 7, wherein the sliding support comprises:
a vertical groove;
a driving rotating wheel driven to rotate by a third motor, and
a synchronous belt fixedly attached to the movable seat, which is driven to rotate by the driving rotating wheel, wherein the movable seat is configured to move in the vertical groove with movement of the synchronous belt; and
a driven rotating wheel driven by the synchronous belt, and
the movable seat comprises:
a clamping portion slidably clamped in the vertical groove, and
a transition portion rotatably connected to the forward and backward moving mechanism.

9. The nasal swab sampling robot according to claim 1, wherein the circumferential angle swinging mechanism comprises a circumferential guide rail, a circumferential movable block connected to the vertical moving mechanism, and a rotating gear, and the circumferential movable block is slidable along the circumferential guide rail through the rotating gear.

10. The nasal swab sampling robot according to claim 9, wherein the circumferential guide rail comprises:
an annular groove; and
annular outer edge teeth meshed with the rotating gear driven by a fourth motor, the circumferential movable block comprises:
a connecting seat, wherein the rotating gear is fixedly connected with the sliding block via the connecting seat, and the connecting seat is driven by the rotating gear to move along the annular outer edge teeth; and
a sliding block, which is driven to slide in the annular groove with movement of the connecting seat.

11. The nasal swab sampling robot according to claim 9, a guide rail supporting base and a counterweight supporting base are further provided, the circumferential guide rail is fixed on the guide rail supporting base, the counterweight supporting base is fixed under the guide rail supporting base, and a ratio of a bottom area of the counterweight supporting base to a bottom area of the guide rail supporting base ranges from 1.5 to 3.

12. The nasal swab sampling robot according to claim 1, wherein a positioning mechanism comprising a limiting frame and a vision device is further provided, the limiting frame is hollowed out in a length direction of the nasal swab sampling head to restrict the vertical displacement of the nasal swab sampling head, and the vision device is provided on the limiting frame to accurately position the nasal swab sampling head.

13. A sampling method conducted by the nasal swab sampling robot according to claim 1, comprising steps of:
S1) determining, by a vision device, whether a patient is present in front of the sampling robot, if not, maintaining the sampling robot in a powered-off state, and if yes, switching the sampling robot to a standby state;
S2) determining, by the vision device, a nostril position to be sampled according to images of patients of different heights and ages in front of the robot, and sending an instruction to control the forward and backward moving mechanism, the vertical moving mechanism, and the circumferential angle swinging mechanism to perform a rough adjustment at entrance of the nostril position;
S3) determining, by the vision device, a nasal bridge position to be sampled according to the images, calculating an inclination angle of the nostril close to the nasal bridge position, and automatically sending an instruction to control the pitch angle swinging mechanism to perform a rough adjustment in a direction parallel to the inclination angle;
S4) inserting the nasal swab sampling head into the nostril after the rough adjustment, and sending a feedback instruction to the pitch angle swing mechanism, the forward and backward moving mechanism, and the circumferential angle swing mechanism to perform a fine adjustment according to resistance experienced by the force feedback member in a nasal cavity; and
S5) performing sampling upon reaching a sampling position in the nasal cavity, and withdrawing from the nasal cavity along the direction parallel to the inclination angle in S3 after sampling is completed.
